(19) United States
(12) Patent Application Publication
(10) Pub. No.: US 2009/0318948 A1
(43) Pub. Date: Dec. 24, 2009

(54) DEVICE, SYSTEM AND METHOD FOR ANEURYSM EMBOLIZATION

(75) Inventors: Richard J. Linder, Sandy, UT (US); Scott D. Miles, Sandy, UT (US); Daryl R. Edmiston, Draper, UT (US); Clark C. Davis, Holladay, UT (US)

Correspondence Address: Holland & Hart LLP/Coherex Medical, 60 East South Temple, Suite 2000, Salt Lake City, UT, 84111, US

(73) Assignee: COHEREX MEDICAL, INC., Salt Lake City, UT (US)

(21) Appl. No.: 12/428,360

(22) Filed: Apr. 22, 2009

Related U.S. Application Data
(60) Provisional application No. 61/047,058, filed on Apr. 22, 2008.

(51) Int. Cl. A61M 29/00

(52) U.S. Cl. 606/191

(57) ABSTRACT

An apparatus, method and system directed to treatment of aneurysms are disclosed. In one embodiment a medical device delivery system may include a handle, a controller coupled to the handle and a catheter coupled to the handle. The delivery system may also include multiple embolic elements. Each of the embolic elements are positioned in a distal portion of the catheter in a compressed configuration and lined in a row within the distal portion of the catheter. The embolic elements are configured to be separately and discretely released from the catheter to be freely and randomly positioned within an aneurysm cavity and are each configured to self expand to an configuration larger in size than the compressed configuration.

### CROSS-REFERENCE TO RELATED APPLICATIONS

The present application claims the benefit of U.S. Provisional Patent Application Ser. No. 61/047,058, filed Apr. 22, 2008, entitled DEVICE AND SYSTEM FOR ANEURYSM EMBOLIZATION, the disclosure of which is incorporated by reference herein in its entirety.

### TECHNICAL FIELD

The present invention relates generally to methods, devices and systems for interventionally occluding body cavities. More particularly, embodiments of the present invention are described in relation to methods, devices and systems for creating an embolism within an aneurysm and the like.

### BACKGROUND

Occlusion of various types of body cavities and lumens by embolization is often desired in a number of clinical situations. For example, the repair of various cardio vascular defects, such as, patent foramen ovale, patent ductus arteriosis, left atrial appendage, and atrial septal defects, are treated with interventional methods and include various embolization techniques. Another example is occlusion of the fallopian tubes for sterilization purposes. Further, for some time now, vascular embolization has been used to control vascular bleeding, to occlude the supply of blood to tumors, and to occlude vascular aneurysms. Such treatment of aneurysms via vascular embolization has received much attention and, as such, many methods and systems have been developed for such aneurysm treatment.

Treatment of aneurysms has included such methods as inflating a balloon with a solidifying gel within the aneurysm, the direct injection of a liquid polymer agent into the desired site, and the use of so-called micro coils. The use of micro coils includes placing a coil of material (e.g., a biocompatible metal or a polymer) within the aneurysm to fill its volume. The micro coils may also include a fiber material, such as a polyester material, to promote thrombosis within the aneurysm. Such methods, and others, have seen varied success in practice.

There is a continuing need in the art to develop devices and methods that are efficient and effective in treating aneurysms. Embodiments of the present invention are described herein with regard to devices, systems and methods for occluding, for example, an aneurysm through embolization.

### BRIEF SUMMARY OF THE INVENTION

Certain embodiments of the present invention are directed to methods, devices and systems for creating an embolism within an aneurysm and the like. In one particular embodiment, a medical device system is provided. The system comprises a handle and a catheter coupled to the handle. A plurality of embolic elements is positioned in a distal portion of the catheter in a compressed configuration. The embolic elements are configured to be separately and discretely released from the catheter to be freely and randomly positioned within an aneurysm cavity. Each embolic element is configured to self expand to an expanded configuration larger in size than the compressed configuration.

In one embodiment, the system may further include a tubular stent having a frame defining a plurality of open cells. The catheter may further include a discharge opening that is sized and configured to extend through at least one of the cells of the plurality of open cells. Additionally, each embolic element, when in the expanded configuration, may exhibit a volume of sufficient size to prohibit passage of the embolic element through any cell of the plurality of open cells.

In accordance with another embodiment of the invention, a method is provided for treating an aneurysm with a multi-cellular tubular stent positioned adjacent the aneurysm. The method includes inserting a distal portion of a catheter in a vessel and positioning the distal portion of the catheter adjacent the aneurysm. A distal tip of the catheter is inserted through a cell of the tubular stent and into an aneurysm cavity. A plurality of discrete embolic elements is deployed from the distal portion of the catheter and into the aneurysm cavity, wherein each of the plurality of embolic elements self expand to a size larger than the cell of the tubular stent.

In accordance with yet another embodiment of the present invention, a medical device is provided that is configured to be positioned within an aneurysm through a multi-cellular tubular stent positioned adjacent the aneurysm. The medical device comprises a plurality of discrete embolic elements, each embolic element being configured to self expand from a first size to a second size, the second size being larger than cells of the multi-cellular tubular stent positioned adjacent the aneurysm.

### BRIEF DESCRIPTION OF THE SEVERAL VIEWS OF THE DRAWINGS

The foregoing and other advantages of the invention will become apparent upon reading the following detailed description and upon reference to the drawings in which:

FIG. 1 is schematic side view of a distal portion of a medical device delivery system and a tubular stent each positioned adjacent an aneurysm, depicting the delivery system deploying multiple separate and discrete embolic elements into an aneurysm cavity through a cell of the tubular stent, according to an embodiment of the present invention;

FIG. 1A is a side view of a medical device delivery system, according to an embodiment of the present invention;

FIG. 2A is a cross-sectional view of one embodiment of a distal portion of the delivery system, depicting the distal portion including a catheter and an inner lumen with a pusher member and multiple embolic elements disposed within the inner lumen, according to the present invention;

FIG. 2B is a cross-sectional view of the delivery system of FIG. 2A, depicting the deployment of an embolic element from the catheter at one state;

FIG. 2C is a cross-sectional view of the delivery system of FIG. 2A, depicting deployment of an embolic element from the catheter at another state;

FIG. 2D is a cross-sectional side view of the delivery system of FIG. 2A, depicting deployment of an embolic element from the catheter;

FIG. 3 is a cross-sectional side view of a distal portion of the inner lumen depicted in FIG. 2A, according to an embodiment of the present invention;

FIG. 4A is a perspective view of a distal end of a portion of the delivery device depicted in FIG. 2B according to an embodiment of the present invention;

FIG. 4B is a perspective view of a distal end of a portion of the delivery device FIG. 2A according to an embodiment of the present invention;

FIG. 5A is a cross-sectional side view of a distal portion of a delivery system including a pusher member proximal multiple embolic elements within a catheter, according to another embodiment of the present invention;

FIG. 5B is a cross-sectional side view of the delivery system of FIG. 5A depicting the pusher member forcing a distal most embolic element from the catheter, according to an embodiment of the present invention;

FIG. 6A is a cross-sectional side view of another embodiment of a distal portion of a delivery system including a pusher member proximal multiple embolic elements with a skewer member positioned through the multiple embolic elements, according to the present invention;

FIG. 6B is a cross-sectional side view of the delivery system of FIG. 6A depicting the pusher member forcing a distal most embolic element from the catheter and from an end of the skewer, according to an embodiment of the present invention;

FIG. 7 is a cross-sectional side view of another embodiment of a delivery system, depicting a distal portion of the delivery system having a conveyer arrangement with a moveable member configured to convey embolic elements from an inner lumen of the delivery system, according to the present invention; and

FIG. 8 is a cross-sectional side view of another embodiment of a delivery system, depicting a pusher member disposed within a catheter to push an embolic element therefrom, according to the present invention.

### DETAILED DESCRIPTION OF THE INVENTION

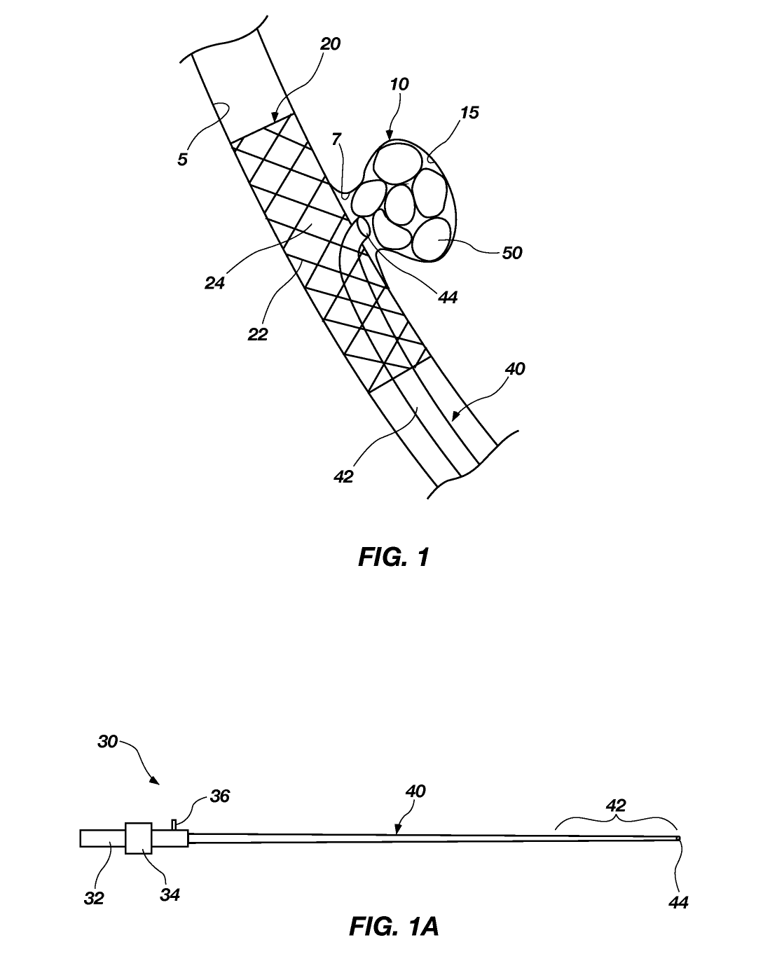

Referring first to FIG. 1, there is shown a distal portion 42 of a catheter 40 of a medical device delivery system 30 (see FIG. 1A) configured to deliver separate, discrete and unconnected embolic elements 50 to an aneurysm 10 and, more specifically, into an aneurysm cavity 15. In one embodiment of the present invention, a tubular stent 20 may be positioned in a vessel 5 such that a portion of the stent 20 is positioned over, or extends across, an opening 7 of the aneurysm cavity 15. The stent 20 may include a tubular frame member 22 configured to define a multi-cellular structure, and may be employed in certain embodiments of the present invention, as a retainer member. Thus, the stent 20 may remain in the vessel 5 after delivery of the embolic elements 50 to the aneurysm cavity 15.

A plurality of open cells 24, defined by the frame member 22 (or members) may be sized and configured so as to facilitate a distal end portion 44 of a catheter 40 of the medical device system 30 to be inserted through a cell 24 of the frame member 22 and into the aneurysm cavity 15. In other words, the distal portion 42 of the catheter 40 may extend into an interior volume defined by the tubular stent 20, with the end portion 44 extending through one of the plurality of cells 24 towards, or even into, the aneurysm cavity 15.

The medical device delivery system 30 is configured deploy a plurality of separate, discrete and unconnected embolic elements 50 within the aneurysm cavity 15. The embolic elements 50 are in a compressed configuration while disposed within the catheter 40 and, when released from the catheter, may expand to a desired size. In one embodiment, each of the embolic elements 50 are separately and discretely released from the catheter 40 to migrate in a free and random manner within the aneurysm cavity 15. Further, according to an embodiment of the present invention, the embolic elements 50 may be configured so as to self expand once when they released from the distal end portion 44 of the catheter 40. For example, the embolic elements 50 may expand to a size greater than the opening of the cells 24 of the frame member 22. Thus, once expanded, the stent 22 serves to prevent the embolic elements 50 from migrating out of the aneurysm cavity 15. In one embodiment, the embolic elements 50 may expand to a volume that is approximately two to three times larger than the volume of their respective compressed configurations.

In certain embodiments, the expanded volume of the embolic elements 50, or the ratio of expanded volume compared to compressed volume of the embolic elements 50, largely depends on the material being used for the embolic element 50. For example, polyurethane foam can expand two to three times larger and up to approximately six times larger the volume of their compressed configuration. In other embodiments, the embolic elements 50 may be configured to expand to even greater relative volumes, for example, use of a polyvinyl alcohol (PVA) foam can expand up to sixteen times larger than its compressed configuration.

The delivery system 30 may deploy one or more of the embolic elements 50 until the aneurysm cavity is sufficiently full of the embolic elements 50. In this manner, the stent 20 acts as a retainer member to retain the expanded embolic elements 50 within the aneurysm cavity 15. It is noted that, while the embolic elements 50 may be sized, in their expanded configured, such that they may not pass through a cell of the stent 22, in one embodiment, they may be small enough that, without the stent 22 placed within the vessel 5, such might be able to pass through the cavity opening 7 depending on the particular geometry and characteristics of the aneurysm 10. In another embodiment, the embolic elements 50 might be sized, when in the expanded configuration, such that they may not pass through the cavity opening 7.

When the aneurysm cavity 15 is filled with embolic elements 50, blood flow will be limited to the aneurysm cavity 15 and the embolic elements 50 induce embolization within the aneurysm cavity 15.

The embolic elements 50 may exhibit a variety of shapes or geometries. For example, they may exhibit a spherical shape, a cylindrical shape or any other suitable shape. Further, the embolic elements 50 may be formed as a substantially solid structure, as a generally hollow structure, or as a partially hollow structure. In one embodiment the embolic elements 50 may be formed with a middle or central portion removed to enable greater compression of the embolic elements 50 while also maintaining the size to which the embolic elements 50 can expand. One example of a hollow or partially hollow structure may include a substantially cylindrical annulus.

In one particular embodiment of the invention, the embolic elements 50 may be formed of a material that enables the above-described self expansion without the need of a fluid being present. Thus, for example, the embolic elements may expand on their own, and not because of the presence of a fluid such as blood or a saline solution. In other embodiments, exposure of the embolic elements 50 to a fluid, such as blood, may activate or otherwise effect expansion of the embolic elements 50.

The embolic elements 50 may be made from a variety of materials including, for example, polymeric materials, metallic materials, metallic alloys or combinations thereof. The embolic elements 50 may include a porous material, such as foam (reticulated or non-reticulated), mesh, fabric, felt or any other suitable material having a porous structure that enables the embolic element 50 to be in a small constrained configuration as well as a self-expanded larger configuration that induces embolization within the aneurysm cavity 15.

Examples of more specific materials that the embolic elements 50 may be formed from include, but are not limited to, polyurethane, polyvinyl alcohol (PVA), polytetrafluoroethylene (PTFE, also known as Teflon(g), expanded polytetrafluoroethylene (EPTFE), polyester, silicone, polyethylene terephthalate (PET, also know as Dacron®), titaniumn, stainless steel, NiTi, copper or copper alloys, composites, and combinations thereof. Additionally, other suitable materials, such as a drug induced substance in combination with the above, may be used to induce embolization as known to one of ordinary skill in the art. Also, biodegradable or bioabsorbable polymers that induce embolization may also be used, such as, polylactide (PLA), poly-L-lactide (PLLA), poly-E-caprolactone (PCL) or polyglycolide (PGA).

It is also contemplated that the embolic elements 50 may include a marker. For example, the embolic elements 50 may be impregnated or coated with a desired material to enable a practitioner to view the placement and position of the embolic element 50 within the aneurysm cavity 15, as well as within the delivery system 30, utilizing conventional imaging techniques. Such a marker may be formed, for example, from a radio-opaque material, such as tantalum, gold, platinum or alloys thereof, or from any other suitable radio-opaque material, such as barium sulfate, as is known in the art.

Referring briefly to FIG. 1A, the medical device delivery system 30 is shown according to an embodiment of the present invention. The medical device delivery system 30 is sized and configured to traverse within a vessel 5 toward an aneurysm 10 and controllably deploy embolic elements 50 within the aneurysm cavity 15 (see, e.g., FIG. 1). The medical device delivery system 30 may include, among other things, a handle 32 with a controller 34 interconnected thereto, and a catheter 40 extending from a distal end of the handle 32. The handle 32 may also include a port 36, in communication with the catheter 40, configured to flush the catheter 40 with fluid. Further, at a proximal portion of the handle 32, there can be a loading portion for loading the embolic elements 50 to a distal portion 42 of the catheter 40. For example, U.S. Provisional Application No. 61/143,360 entitled MEDICAL DEVICE FOR MODIFICATION OF LEFT ATRIAL APPENDAGE AND RELATED SYSTEMS AND METHODS, filed Jan. 8, 2009 (the disclosure of which is incorporated by reference herein in its entirety), discloses one means of loading compressible members into medical device for delivery through a catheter. It is also contemplated that the embolic elements 50 can be loaded directly into the catheter 40.

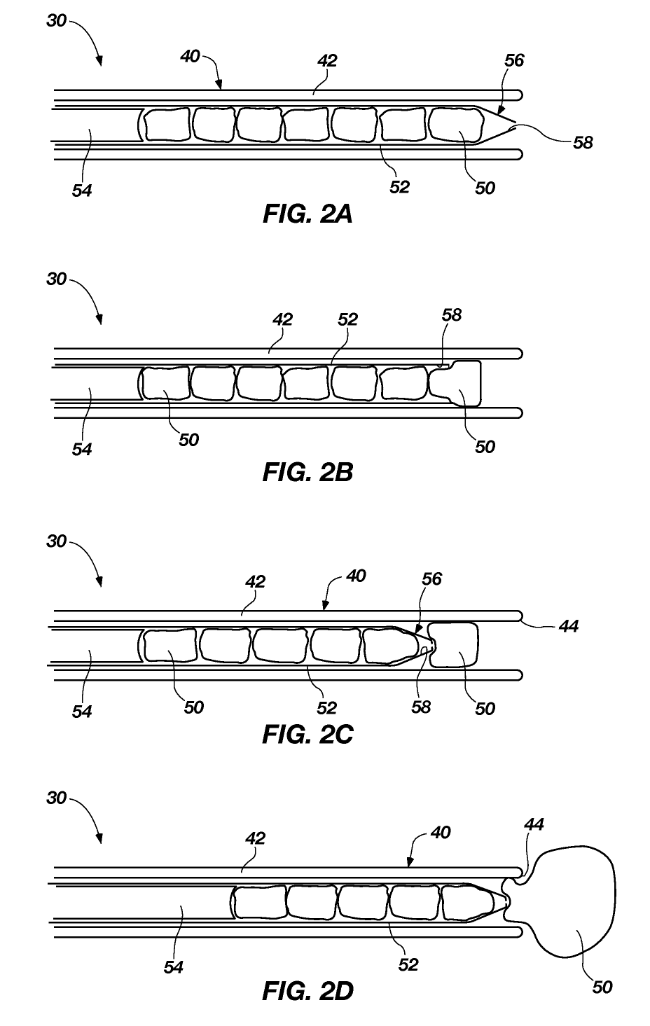

Further, the controller 34 can be configured to manipulate and control the delivery and deployment of the embolic elements 50 from a distal portion 42 of the catheter 40 such as by controlling displacement of various components of the delivery system 30 (e.g., a push rod 54, an inner housing 52, described in reference to FIGS. 2A-2D below).

Referring now to FIGS. 2A through 2D, an embodiment of the delivery system 30 and associated method is disclosed. Each of FIGS. 2A through 2D show a different time or state within a sequence of acts associated with deploying an embolic element 50 from the delivery system 30. In one embodiment, the embolic elements 50 may de deployed or discharged from the distal portion 42 of the delivery system 30 in a ratcheting manner as will be detailed hereinbelow.

With respect to FIG. 2A, a distal portion 42 of the catheter 40 of the delivery system 30 is shown with a plurality of embolic elements 50 disposed therein. An inner housing 52 is positioned within a lumen of the catheter 40. A pusher member or push rod 54 is disposed within a lumen defined by the inner housing 52 and positioned proximally of the plurality of discrete embolic elements 50, the embolic elements 50 being positioned within a distal portion of the lumen defined by the inner housing 52. The inner housing 52 includes a distal tip 56 with a mouth 58 that is moveable between a partially closed (or, in another embodiment, a fully closed) position and an open position. In one embodiment, the partially closed position is the naturally disposed position of the mouth 58 of the distal tip 56. The mouth 58 of the distal tip 56 may be placed in the open position when appropriate force is applied thereto. For example, a force may be applied to the mouth 58 of the distal tip 56 by way of a distal-most embolic element 50 that is being pushed and displaced distally (i.e., to the right in FIGS. 2A-2D) by way of the push rod 54.

In one embodiment, the push rod 54 may include a coil (with a plug at the distal end) formed from, for example, one or more stainless steel wires, or any other suitable pusher member that resists compression and provides a high degree of flexibility, such as a polymeric braided tube or the like. Additionally, in certain embodiments the inner housing 52 can be a tube formed from a polymeric or nitinol material.

FIG. 2B shows an embolic element 50 as it is being deployed from the inner housing 52. In one example, the push rod 54 may remain stationary while the inner housing 52 is displaced proximally (i.e., to the left in FIGS. 2A-2D). As the inner housing 52 is displaced proximally, the mouth 58 of the distal tip 56 opens (due to the force applied to it via the distal-most embolic element 50) such that the distal-most embolic element 50 begins to be deployed from the inner housing 52.

In another embodiment, the push rod 54 may be moved distally while the inner housing 52 either remains stationary or is moved proximally. In any case, the mouth 58 of the distal tip 56 is moved to the open position and the distal-most embolic element 50 begins to be deployed or discharged from the inner housing 52.

Referring to FIG. 2C, the delivery system 30 is shown in another state, or at another time during the sequence of deploying or discharging an embolic element 50. As compared to that which is shown in FIG. 2B, the inner housing 52 has now been displaced proximally with respect to the embolic elements 50 so that the mouth 58 of the distal tip 56 is moved proximal of the embolic element 50 that has just been deployed from the inner housing 52. The mouth 58 of the distal tip 56 now returns to its preferentially closed (or partially closed) state as seen in FIG. 2C. In this state, the inner housing 52 (as well as the push rod 54 in some embodiments) may be displaced distally to push the embolic element 50 deployed from the inner housing 52 (but still within the catheter 40) distally within the catheter. The inner housing 52, with the mouth 58 of the distal tip 56 in the closed or partially closed position, thus acts as pusher member against the proximal side of an embolic element 50 that has been deployed from the inner housing 52.

Referring now to FIG. 2D, the embolic element 50 previously deployed from the inner housing 52 is shown while being deployed from the catheter 40. As described above, the inner housing 52 (along with the push rod 54 in some embodiments) is displaced distally to push an embolic element 50 (previously deployed from the inner housing) from the distal end portion 44 or opening of the catheter 40. With such distal movement of the inner housing 52, the remaining embolic elements 50 disposed within the inner housing 52 move concurrently with the inner housing 52 until the embolic element within the catheter is pushed distally from the catheter 40. Once the embolic element 50 is free of the catheter 40, the released or deployed embolic element 50 self expands. The inner housing 52, with the compressed, constrained embolic elements 50 disposed therein, is positioned again as depicted in FIG. 2A and the sequence may be repeated to deploy another embolic element. In such a configuration, the embolic elements 50 can be sequentially and consecutively dispersed from the catheter 40 into the aneurysm cavity 15 in a controlled manner.

It is also noted that the inner housing 52 may be configured for removal from the medical device system 30, such as by withdrawing it through the handle. In such a case, if all of the embolic elements 50 disposed within the inner housing 50 had been deployed into an aneurysm 10, and the aneurysm 10 still was not satisfactorily filled or occluded, a new inner housing 52, pre-loaded with embolic elements 50, could be inserted into the medical device system 30 such that additional embolic elements 50 could be delivered through the catheter 40 without removing the catheter from the patient.

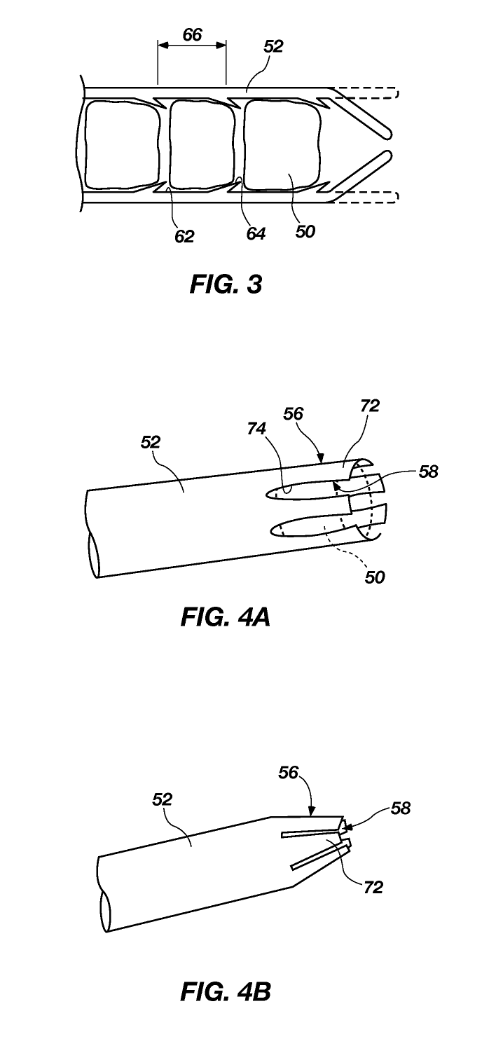

Referring now to FIG. 3 further details of the inner housing 52, such as depicted in FIGS. 2A through 2D, are shown in accordance with an embodiment of the present invention. The inner housing 52 may include an inner surface 62 having protrusions 64 extending distally and slightly radially inward. The protrusions are configured to facilitate substantially unidirectional distal movement of the embolic elements 50 within the inner housing 52. Such protrusions 64 may be positioned in a predetermined manner along the longitudinal length of the distal portion of the inner housing 52. For example, protrusions 64 may be longitudinally space a length 66 between that corresponds with a length (or slightly longer than a length) of an individual embolic element 50 disposed within the inner housing 52. Such protrusions 64 may include a substantially annular configuration (i.e., the may extend substantially about the internal periphery or circumference of the inner housing 52 in a ring-like manner). In another embodiment, the protrusions 64 may extend from the inner housing 52 in a partially annular manner or, in another embodiment, they may simply include discrete protrusions located at specific points along the inner surface 62.

The protrusions 64 within the inner housing 52 enable distal movement of the embolic elements 50 and prevent substantial proximal movement of the embolic elements 50 when deploying the embolic elements utilizing, for example, the method of deploying the embolic elements 50 as depicted in FIGS. 2A through 2D, such that the embolic elements 50 advance in a ratcheting-like manner. In other words, the protrusions act as a sort of mechanical check valve for the embolic elements 50. In another embodiment, the protrusions 64, while still being oriented to extend in the distal and radially inward directions, may be positioned randomly along the inner surface 62 of the inner housing 52.

Referring now to FIGS. 4A and 4B, perspective views of the mouth 58 at the distal tip 56 of the inner housing 52 with the mouth 58 being shown in both the open position (FIG. 4A) and the partially closed position (FIG. 4B). Referring first to FIG. 4A, the mouth 58 is in the open position with an embolic element 50 (shown as dashed lines) within the mouth 58. As shown in the open position, the mouth 58 may include multiple extensions 72 or segments extending distally from the inner housing 52. The extensions 72 define multiple slots 74 positioned between adjacent extensions 72. The extension 72 and slot 74 arrangement can be configured to enable the mouth 58 to move to the open position (such as by elastically deforming or displacing the extensions 74) as an embolic element 50 is being moved distally from the inner housing 52 through the mouth 58 (see FIG. 2B).

As depicted in FIG. 4B, the mouth 58 is in the partially closed position. This position is employed when an embolic element 50 is not disposed in the mouth 58 (e.g., as shown in FIGS. 2A, 2C and 2D). As previously set forth, the mouth 58 is configured to naturally move to the partially closed position. In other words, the distal ends of the extensions 72 naturally extend radially inward when no external force is applied thereto. The inner housing 52 (including the extensions 74) may be made, for example, from a polymeric material and may be molded using traditional injection molding techniques. In other embodiments, the inner housing 52 (and associated extensions 74) may be made from some other suitable material, such as a metal, a metal alloy, or a shape memory alloy, using an appropriate manufacturing technique.

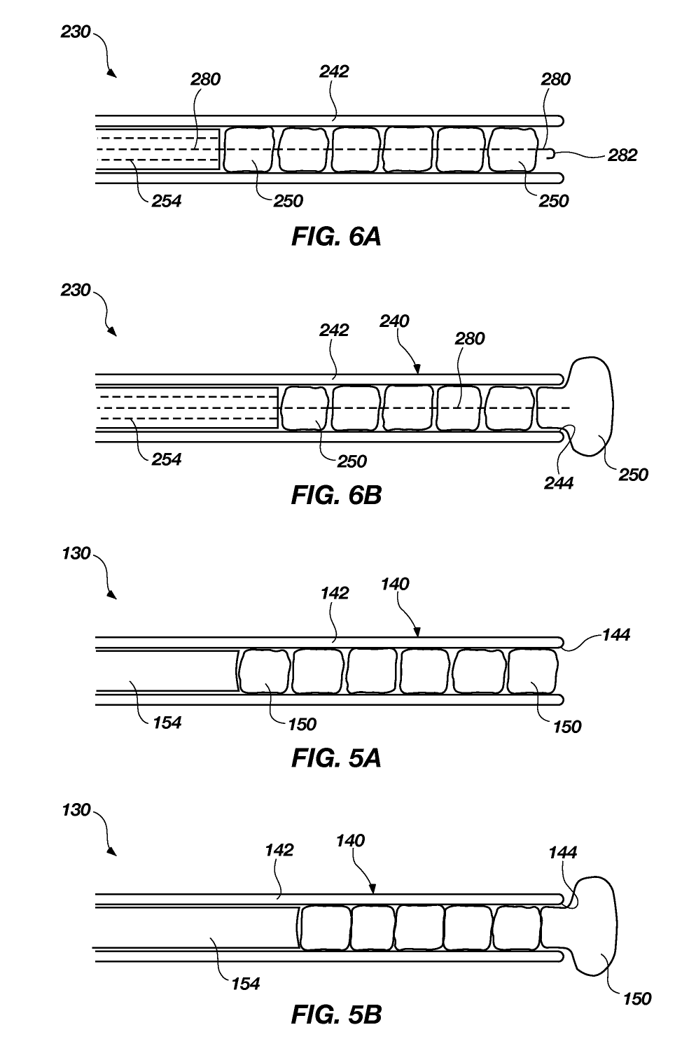

Referring now to FIGS. 5A and 5B, a distal portion 142 of a delivery system 130 is disclosed in accordance with another embodiment of the present invention. As shown in FIG. SA, the delivery system 130 may include, among other things, a catheter 140, a pusher member or push rod 154, and multiple embolic elements 150 compressed within the distal portion of the catheter 140. The push rod 154 is positioned proximally of the embolic elements 150 (i.e., to the left of the embolic elements 50 as shown in FIGS. 5A and 5B) with the embolic elements 50 individually and separately compressed in a sequential line between the push rod 154 and a distal opening 144 defined at the distal end portion of the catheter 140. As depicted in FIG. 5B, the embolic elements 150 can be individually deployed with the push rod 154 moving distally against a proximal most embolic element 150, pushing forward toward the distal opening 144 to, thereby, force the distal most embolic element 150 from the distal opening 144 at the distal end portion of the catheter 140. The push rod 154 can continue to move distally to push or force additional embolic elements 150 from the distal opening 144 of the catheter 140. With this arrangement, the delivery system 130 can deploy embolic elements 150 within an aneurysm cavity, similar to that depicted in FIG. 1 and FIGS. 2A-2D.

Referring to FIGS. 6A and 6B, a distal portion 242 of a delivery system 230 is disclosed according to another embodiment of the present invention. Referring first to FIG. 6A, this embodiment is substantially similar to the embodiment described with respect to FIGS. 5A and 5B, except this embodiment includes a skewer member 280 that may be in the form of a rod or line. The skewer member 280 extends through each of the embolic elements 250 and through the push rod 254 or other pusher member. The skewer member 280 may be fixed at a proximal end thereof (not shown) and may be sized and configured to provide structural support to the embolic elements 50 and to maintain and control the embolic elements 250 in a lined fashion. The skewer member 280 may include a distal free end 282, wherein the distal free end 282 can include a curved portion so as to prevent the embolic elements 250 from self migrating relative to the skewer member extending through each of the embolic elements 250.

Referring to FIG. 6B, deployment of an embolic element 250 may be accomplished by displacing the push rod 254 distally against the proximal-most embolic element 250. As in the previous embodiment, distal movement of the push rod 254 relative to the catheter 240 results in a chain reaction of forces that pushes the most distal embolic element 250 from a distal opening 244 of the distal portion 242 of the catheter 240. As the distal-most embolic element 250 moves toward the distal opening 242 and over the curved portion of the skewer member 280, the curved portion temporarily straightens to enable the distal-most embolic element 250 to be deployed from the catheter 240.

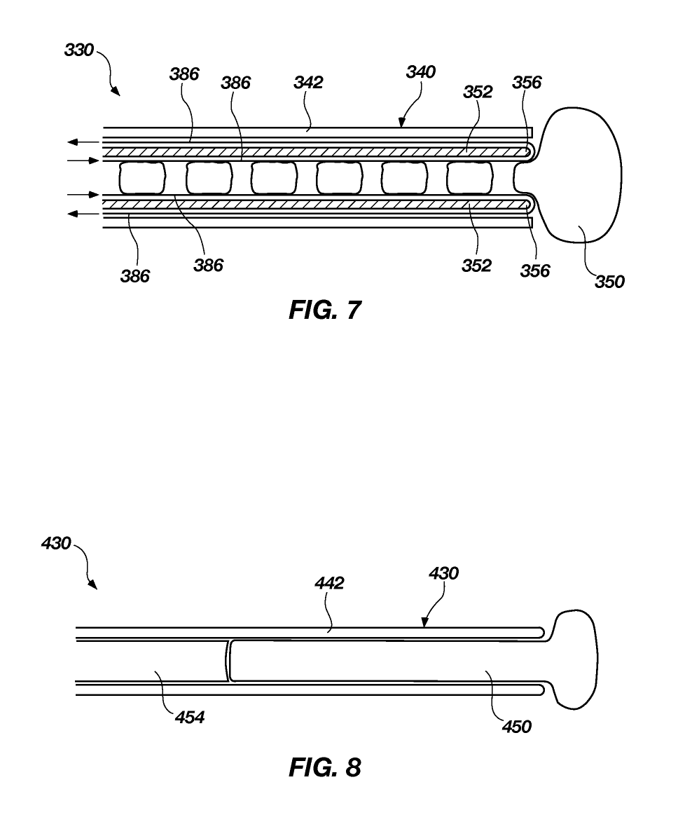

Referring now to FIG. 7, the distal portion 342 of another delivery system 330 is shown. As with previously described embodiments, the delivery system 330 is configured to deploy embolic elements 350 sequentially and in a separate, discreet and unconnected manner from a distal portion 342 thereof. In the presently considered embodiment, the delivery system 330 includes a catheter 340, an inner housing 352 disposed within a lumen of the catheter 340, and a moveable member 386 in direct contact with the embolic elements 350. The inner housing 352 can be in a fixed position. The moveable member 386 may exhibit a generally tubular configuration and be sized and configured to move along a path from within the inner housing 352, around a distal end 356 of the inner housing 352, and to an outer surface of the inner housing 352 between the inner housing 352 and the inner surface of the catheter 340. The embolic elements 350 can be disposed within the inner lumen 352 and, further, within the tubular configuration of the moveable member 386 such that the embolic elements 350 may be moved and dispersed out of the catheter 340 when the moveable member 386 is displaced in the manner described above. In this manner, the moveable member 386 frictionally or otherwise engages the embolic elements 350 and moves them in a conveyer belt-type manner out of the catheter 340.

In one embodiment, the moveable member 386 may be a flexible member formed of, for example, a woven material or a skin-like material sized and configured to move from inside the inner housing 352 to an outer surface of the inner housing 352. Such a moveable member 386 can also expand so as to allow lateral widening around a tip of the inner lumen 352. In another embodiment, the moveable member 386 may include a plurality of longitudinally extending lines. Such moveable member 386 can be made from, for example, a polymeric material or Nitinol.

FIG. 8 discloses another embodiment for deploying an embolic element 450 from a distal portion 442 of a delivery system 430 to and within an aneurysm cavity. In this embodiment, the delivery system 430 may include a catheter 440 with a pusher member or push rod 454 positioned proximally of an embolic element 450. In the presently considered embodiment, the embolic element 450 may be elongated and cylindrical in shape with a worm-like configuration. Similar to the previous embodiments, such embolic element 450 is self expanding and can be made from, for example, a foam or foam-type material. Further, the delivery system 430 may include a cutting element (not shown) at a distal end of the catheter so that the embolic element 450 can be cut or sliced once the embolic element has satisfactorily filled the aneurysm. Alternatively, a plurality of smaller discrete embolic members may be cut from the elongated embolic element to fill an aneurysm cavity such as has been described with respect to other embodiments.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention includes all modifications, equivalents, and alternatives falling within the spirit and scope of the invention as defined by the following appended claims.

What is claimed is:

1. A medical device system, comprising:
a handle;
a catheter coupled to the handle; and
a plurality of embolic elements positioned in a distal portion of the catheter in a compressed configuration, the plurality of embolic elements being configured to be separately and discretely released from the catheter to be freely and randomly positioned within an aneurysm cavity, each embolic element being configured to self expand to an expanded configuration larger in size than the compressed configuration.

2. The system of claim 1, wherein each embolic element, when in the expanded configuration, exhibits a volume that is at least twice as large as a volume of the compressed configuration.

3. The system of claim 1, wherein each embolic element, when in the expanded configuration, exhibits a volume that is at least three times as large as a volume of the compressed configuration.

4. The system of claim 1, wherein each embolic element is formed of a material comprising at least one of polyurethane, expanded polytetrafluoroethylene (EPTFE), polyvinyl alcohol (PVA), polytetrafluoroethylene (PTFE), polyester, silicone, polyethylene terephthalate (PET), titanium, stainless steel, NiTi or copper.

5. The system of claim 1, further comprising a tubular stent having a frame defining a plurality of open cells.

6. The system of claim 5, wherein the catheter includes a discharge opening sized and configured to extend through at least one of the cells of the plurality of open cells.

7. The system of claim 5, wherein the each embolic element, when in the expanded configuration, exhibits a volume of sufficient size to prohibit passage of the embolic element through any cell of the plurality of open cells.

8. The system of claim 1, further comprising an inner housing disposed within a lumen of the catheter, wherein the plurality of embolic elements are disposed within a lumen of the inner housing.

9. The system of claim 8, wherein the inner housing includes a plurality of extensions, each extension being separated from an adjacent extension by a slot, the plurality of extensions being elastically displaceable from a first position to a second position.

10. The system of claim 8, further comprising a plurality of distally and radially inward extending protrusions formed on an inner surface of the inner housing.

11. The system of claim 10, wherein the protrusions are spaced in correlation with a length of individual embolic elements of the plurality of embolic elements.

12. The system of claim 1, further comprising a push rod movably disposed within the catheter and configured to place a force on the plurality of embolic elements.

13. A method for treating an aneurysm with a multi-cellular tubular stent positioned adjacent the aneurysm, the method comprising:
inserting a distal portion of a catheter in a vessel;
positioning the distal portion of the catheter adjacent the aneurysm;
inserting a distal tip of the catheter through a cell of the tubular stent and into an aneurysm cavity; and
deploying a plurality of discrete embolic elements from the distal portion of the catheter and into the aneurysm cavity, each of the plurality of embolic elements self expanding to a size larger than the cell of the tubular stent.

14. The method according to claim 13, further comprising substantially filling the aneurysm with the plurality of discrete embolic elements.

15. The method according to claim 13, further comprising configuring the plurality of embolic elements to expand from a first volume to a second volume that is at least approximately twice as large as the first volume.

16. A medical device configured to be positioned within an aneurysm through a multi-cellular tubular stent positioned adjacent the aneurysm, the medical device comprising:
a plurality of discrete embolic elements, each embolic element being configured to self expand from a first size to a second size, the second size being larger than cells of the multi-cellular tubular stent positioned adjacent the aneurysm.

17. The medical device of claim 16, wherein the second size is a volume that is at least twice as large as a volume of the first size.

18. The medical device of claim 16, wherein the second size is a volume that is at least three times as large as a volume of the first size.

19. The medical device of claim 16, wherein each embolic element exhibits a substantially cylindrical geometry.

20. The medical device of claim 16, wherein each embolic element is formed of a material comprising at least one of polyurethane, expanded polytetrafluoroethylene (EPTFE), polyvinyl alcohol (PVA), polytetrafluoroethylene (PTFE), polyester, silicone, polyethylene terephthalate (PET), titanium, stainless steel, NiTi or copper.
